(19) United States
(12) Patent Application Publication
(10) Pub. No.: US 2023/0000602 A1
(43) Pub. Date: Jan. 5, 2023

(54) Suction Ring

(71) Applicant: Ivoclar Vivadent AG, Schaan (LI)

(72) Inventor: Markus Lichtensteiger, Montlingen (CH)

(21) Appl. No.: 17/807,175

(22) Filed: Jun. 16, 2022

(30) Foreign Application Priority Data
Jul. 1, 2021 (EP) 21183281.1

(51) Int. Cl. A61C 17/10; A61C 17/08

(52) U.S. Cl. CPC A61C 17/10; A61C 17/08

(57) ABSTRACT

A suction ring for mounting close to the mouth opening of a dental patient, comprising an extraoral tensioning element. The tensioning element is realised as a suction tensioning element and forms or spans a negative pressure space with a film element. The negative pressure space extends annularly or partially annularly, and the negative pressure space has at least one suction nozzle, in particular pointing radially inwards.

### CROSS-REFERENCE TO RELATED APPLICATIONS

This application claims priority to European patent application No. 21183281.1 filed on Jul. 1, 2021, which disclosure is incorporated herein by reference in its entirety.

### TECHNICAL FIELD

The present invention relates to a suction ring according to the claims.

### BACKGROUND

Dental devices have been known for a long time. These include saliva ejectors in particular. However, the sucking of aerosols out of the patient's mouth area has also been known for a long time.

An appropriate suction device is intended to prevent the dentist being infected by the patient, but also to improve the absence of germs and viruses in the treatment room.

An example of such a suction device can be found in DE 37 26 394 A1. In this aerosol suction device, a suction nozzle is placed in the mouth area of the patient and, and the aerosols present through and in the patient's breathing air can be sucked off and separated. U.S. Pat. Nos. 4,261,697, 7,300,401, 10,959,820, 5,127,411, 20080153058 and 20220110715 are directed to devices used during dental treatment and are hereby incorporated by reference in their entirety.

Furthermore, the provision of a negative pressure source in the vicinity of the person releasing germs or viruses has already been suggested. This is intended to supply germ-free fresh air from the environment so that infections are prevented.

The disadvantage of such a solution is that it is not guaranteed that the person does not infect other people, especially those who are close to his mouth opening.

### SUMMARY

Therefore, the invention is based on the object of creating a suction ring according to the claims, which offers improved infection safety, but is still easy to handle.

This object is solved according to the invention by the claims. Advantageous further embodiments result from the sub-claims.

According to the invention, it is intended to provide a suction ring for aerosols which can be attached directly to the mouth opening of a patient.

From an infectiousness point of view, the mouth opening regularly represents the source of infection, especially with regard to the release of aerosols.

According to the invention, the suction ring is placed under negative pressure as far as its negative pressure chamber is concerned.

The negative pressure chamber extends slightly outside the mouth opening and places the area of the mouth opening under negative pressure via a suction nozzle. This ensures that germs or viruses released by the patient are sucked out of the mouth immediately after release and thus rendered harmless.

Preferably, the suction nozzle extends radially inwards. However, it is also possible that it extends into the mouth opening or aspirates the area in front of the mouth opening and ends there accordingly.

The term suction ring is to be understood here as any element, in particular a curved element, that can be regarded as at least partially ring-shaped. This means that a closed ring, i.e., a full ring, does not have to be present in order to achieve the desired effect.

It is also possible to perform suction at two points distributed around the circumference at the mouth opening of the patient, or else, at several points distributed around the circumference.

The dimensions of the suction ring according to the invention are preferably such that it can rest extra-orally on the patient's lips, even when the patient has opened his mouth. This results in a cross-section or diameter of about 5 to 10 cm.

In accordance with the invention, the suction ring is provided with a tensioning element and a film element. The film element is connected to the tensioning element.

The film element preferably passes through the mouth opening of the patient in a manner known per se. Thus, the tensioning element is positioned in front of the mouth opening of the patient. The tensioning element has a dual function in that it tensions the film element on the one hand and provides or tensions the negative pressure space on the other.

The film element lies against the patient's lips due to the tensioning effect of the tensioning element, so that contact between the patient's lips and the dentist's hands is also safely excluded.

This function is known from the very successful lip/cheek retainers “Optragate” of the present applicant. Such a holder has an intraoral tensioning element and an extraoral holder tensioning element, between which the film element extends. Such a holder can be used well according to the invention.

The tensioning element according to the invention is e directly connected to the film element as a special extraoral suction tensioning element. However, it is also possible to use a standard holder and, for example, the “Optragate” holder.

The extraoral suction tensioning element according to the invention is then attached to the extraoral holder tensioning element. The attachment can be made in any suitable manner, for example by the moulding forming the tensioning element according to the invention being clipped or clipped on or clamped or clamped on to the holder-tensioning element.

Any suitable connection is possible. Preferably, such a connection, which is free of undercuts and, in particular, does not create any internal corners on the suction tensioning element according to the invention.

The film element lies against the patient's lips due to the tensioning effect of the tensioning element, so that contact between the patient's lips and the dentist's hands is also safely excluded. This function is known from the very successful lip/cheek retainers “Optragate” of the present applicant. US 20220110715, directed to Optragate, is hereby incorporated by reference in its entirety.

Such a holder has an intraoral tensioning element and an extraoral holder tensioning element, between which the film element extends. Such a holder can be used well according to the invention.

The tensioning element according to the invention is directly connected to the film element as a special extraoral suction tensioning element. Alternatively, it is also possible to use a standard holder and, for example, the “Optragate” holder.

The extraoral suction tensioning element according to the invention is then attached to the extraoral holder tensioning element. The attachment can be made in any suitable manner, for example by the moulding forming the tensioning element according to the invention being clipped or clipped on or clamped or clamped on to the holder-tensioning element.

Any suitable connection is possible. Preferably, such a connection, which is free of undercuts and, in particular, does not create any internal corners on the suction tensioning element according to the invention.

Typically, the holder tensioning element is more elastic than the suction tensioning element according to the invention. It therefore makes sense to provide the bearing by elastically deforming the holder tensioning element, which is accordingly pushed onto or into the suction tensioning element under pretension and wedges there.

This solution has the advantage that the suction ring according to the invention can be used several times on standard lip/cheek holders or that such holders can be retrofitted with a suction ring according to the invention.

Both the vacuum chamber according to the invention and the suction nozzle according to the invention can be designed in any suitable manner. The vacuum chamber is equipped with a suction connection. Via this, it is connected to a vacuum source regularly available in dental practices. Preferably, the negative pressure chamber is formed in a plastic body, for example in the form of an injection moulded part.

However, the negative pressure space may also be held taut by one or more tensioning elements which are covered by the foil element or between which a foil element extends. The film element may be a separate foil element intended to form the vacuum space, or it may be integral with the foil element of the lip/cheek retainer, thus extending it extra orally.

According to the invention, it is particularly advantageous that the tensioning element keeps the film element under tension. The film element of the lip/cheek retainer extends through the mouth opening and covers the patient's lips. The pretensioning of the tensioning element pulls the film element there flat so that no wrinkles occur. It is understood that the extraoral tensioning element preferably cooperates with an intraoral tensioning element, as is known with the “Optragate” per se. In this embodiment, the film element connects the extraoral tensioning element with the intraoral tensioning element.

The film element is essentially tubular in a manner known per se, with a central, inwardly curved constriction. It is preferably part of the lip/cheek holder, which also has a circular or oval intraoral tensioning element. This may also be referred to as an intraoral tension ring. Its purpose is to hold the film element taut intraorally in the vestibule so that the film element is in full contact with the patient's lips.

In a known lip/cheek retainer, an extraoral tensioning element or extraoral tensioning ring is also provided, which keeps the film element taut in the same way as the intraoral one. This can be conveniently used as a bearing base of the suction ring. For this purpose, the suction tensioning element is supported on or against the extraoral tensioning ring or element. The bearing can be made by a clamp connection, by a tensioning connection, i.e., one in which the suction tensioning element engages over, under or behind the extraoral tensioning ring and these place each other under tension, or by a clip connection.

Alternatively, it is particularly advantageous if the suction tensioning element holds the film element in the tensioned state. This makes it possible to dispense with an additional extraoral tensioning element of the film element. The tensioning element holds the film element in the tensioned state and is put under tension by the film element itself.

The circular or oval suction tensioning element has a larger diameter than the film element in the relaxed state. By means of a tension connection between them based on this, in which the film element, for example, overlaps the suction tensioning element, both the suction tensioning element is placed under a radially inward tension and the film element is placed under a radially outward tension. This enables a secure but, if necessary, detachable connection.

The suction ring according to the invention is preferably designed for connection to or integral with a lip/cheek holder. This comprises the film element. The film element is designed to extend over the lip of a patient, at which point the film element forms a cover element. In this respect, the suction tensioning element is preferably designed as an extraoral suction tensioning element in such a way that it is connected to the intraoral tensioning element via a covering element as a film element.

In an advantageous embodiment, it is provided that the suction tensioning element has receiving means for the film element, via which it is connected or can be connected to the film element, in particular detachably. A detachable connection is particularly favourable. The suction tensioning element can be designed as a solid plastic body. This receives the vacuum space. The film element can be attached to the suction tensioning element by means of the receiving means.

After use, it is detached and disposed of, and the suction tensioning element can be used for the next treatment.

The receiving means can also be designed to support a lip/cheek holder. This then in turn comprises the film element, so that indirect storage of this is realised by the receiving means of the suction tensioning element.

It is preferable that the suction ring for attachment close to the mouth opening of a dental patient includes an extraoral tensioning element, wherein the tensioning element as a suction tensioning element, optionally in cooperation with a film, forms or spans a vacuum space or chamber, which vacuum space extends annularly or partially annularly, and wherein the vacuum space has at least one suction nozzle or suction zone, pointing radially inwardly.

It is preferable that the suction tensioning element extends annularly around the suction nozzle/zone and/or wherein the suction nozzle extends annularly.

It is preferable that the suction nozzle has a radially extending suction gap or a plurality of radially extending suction nozzles.

It is preferable that the suction nozzle is configured for suction of gases comprising aerosols.

It is preferable that vacuum chamber is fixedly connected to a suction connection for connection to a vacuum source.

It is preferable that the suction tensioning element has a round, circular, oval, or egg-shaped form in a top view from a forward or front side.

It is preferable that the suction tensioning element has a receiving means for the film element, via which it is connected or is configured to be connected, detachably, to the film element.

It is preferable that the film element is firmly connected to the suction tensioning element and/or extends at least partially over the suction tensioning element.

It is preferable that the suction tensioning element holds the film element in the tensioned state, and is placed under tension by the film element itself.

It is preferable that the film element is configured to extend over a lip of a patient, at which point the film element forms a lip-covering element.

It is preferable that the suction tensioning element is designed as an extraoral suction tensioning element which is connected to an intraoral tensioning element via a film element as a lip covering element.

It is preferable that the suction tensioning element is connected or connectable to a lip/cheek holder or is formed integrally therewith.

It is preferable that a lip/cheek holder is retrofitted with the suction ring.

It is preferable that the vacuum chamber and/or the suction tensioning element is formed as a dimensionally stable plastic body, as an injection moulded part.

It is preferable that the suction tensioning element is formed by or comprises a radially outwardly prestressed wire made of plastic or metal, and/or wherein the suction tensioning element extends spirally, the spirals having different diameters, and/or wherein the suction nozzle is formed between the cover element and the tensioning element, in the last spiral of the tensioning element.

It is preferable that the suction ring is usable as a retrofit part or accessory part for a lip/cheek holder.

### BRIEF DESCRIPTION OF THE DRAWINGS

Further advantages, details and features will be apparent from the following description of several embodiments of the invention with reference to the drawings, wherein:

FIG. 1 is a perspective view from an embodiment of a suction ring according to the invention;

FIG. 2 is a perspective view of the suction ring according to FIG. 1, to which a film element is connected;

FIG. 3 is an enlarged section through the embodiment according to

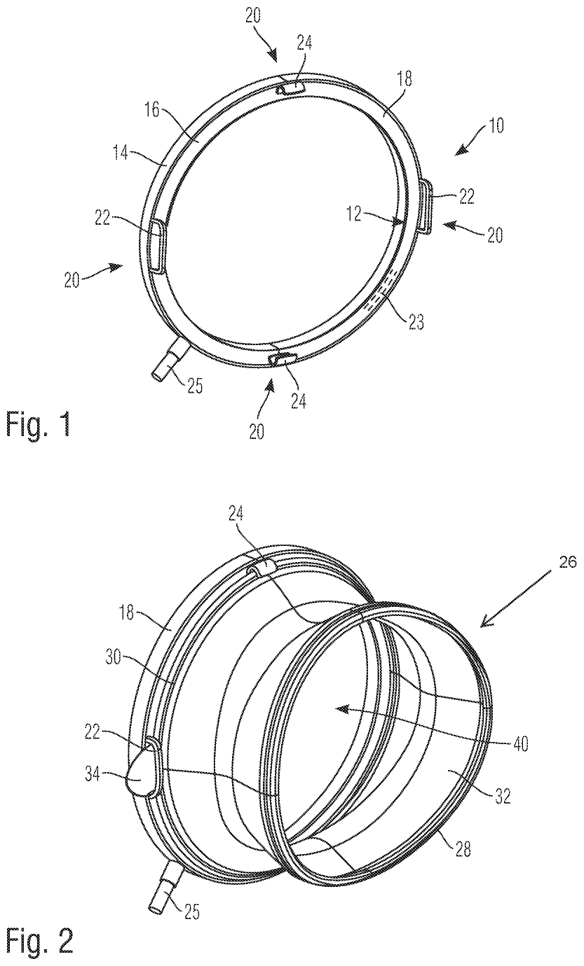

FIGS. 1 and 2, showing the vacuum chamber and the receiving means for connecting the film element to the tensioning element;

FIG. 4 is a schematic representation of an attachment of a suction ring according to the invention, attached to a patient;

FIG. 5 is a schematic perspective view of suction through the suction ring according to the invention;

FIG. 6 is a different representation of the embodiment according to FIG. 5 and with partial removal of a part of the suction ring;

FIG. 7 is a schematic sectional view through a further embodiment of a suction ring according to the invention with integrated lip/cheek holder, with a foil element which is integral with the rest of the suction ring;

FIG. 8 is a schematic top view of the suction ring in the embodiment according to FIG. 7, with a spirally extending tensioning element;

FIG. 9 is a schematic representation of a further embodiment of a suction ring according to the invention in a two-part design with the lip/cheek holder;

FIG. 10 a modified version of the suction ring compared to FIG. 9 using the same lip/cheek holder.

### DETAILED DESCRIPTION

FIG. 1 shows a perspective view of an extraction ring 10 according to the invention. In the illustration according to FIG. 1, the suction ring 10 is circular and designed as a hollow profile. It consists of two injection moulded plastic parts 14, 16 clipped onto each other and has a slot or gap on its radially inner side which forms a suction nozzle 12.

The suction ring 10 is shown in FIG. 1 in a part forming the suction tensioning element 18. The suction tensioning element 18 has receiving means 20. These include two eyelets 22 and two clips 24.

The suction tensioning element 18 spans a vacuum space or chamber 23 which is connected to the suction nozzle 12. The vacuum chamber 23 extends in an annular or partial annular shape, The vacuum chamber 23 is in turn pressurised via a suction connection 25, as this is connected via a hose not shown to a vacuum source not shown.

The suction nozzle 12 is designed as an annular gap and accordingly sucks in air and any aerosols located there from the centre of the suction tensioning element 18. These are collected in the suction tensioning element 18 and discharged via the suction connection 25. Due to the orientation of the suction nozzle 12 radially inwards, the suction from the centre of the tensioning element 18 is particularly favoured.

The receiving means 20 are used to support a lip/cheek holder 26 shown in FIG. 2. The holder 26 has an intraoral tensioning element 28 and an extraoral tensioning element 30, between which a film element 32 extends. By means of the receiving means 20, the holder 26 is mounted on the suction holder tensioning element 18.

In this case, the clamp 24 embraces the extraoral tensioning element 30 from the outside. An outwardly projecting tensioning flap 34 of the holder 26 is inserted into the eyelet 22 and supports it. The tensioning flap 34 is made of foil or film material like the film or foil element 32.

The extraoral tensioning ring 30 is inserted into the suction tensioning ring 18 under pretension. The tensioning ring 30 is compressed somewhat by the action of the clips 24.

The retainer or holder 26 is formed like an “optragate” device in a manner known per se. The tensioning elements 30 and 28 are essentially circular and made of elastic plastic rings. The film element 32 is stretched between them. In the middle between the rings 28 and 30 it is curved radially inwards. This area is intended for the lip support. The film element 32, which is made of a very elastic material, fits snugly against the lips, and extends along them both intraorally and extraorally.

Through the opening 40 in the holder 26 and in the suction tensioning ring 18, the patient can breathe even when the suction ring 10 is in place, and the dentist can perform dental procedures through the opening 40.

When the negative pressure source is switched on, the opening 40 is suctioned at the same time. Breathing air of both the patient and the dentist, if applicable, and aerosols are reliably extracted so that neither the patient nor the dentist are contaminated by each other's breathing air.

This protects both from infections, regardless of whether germs or viruses are present in the breathing air, for example.

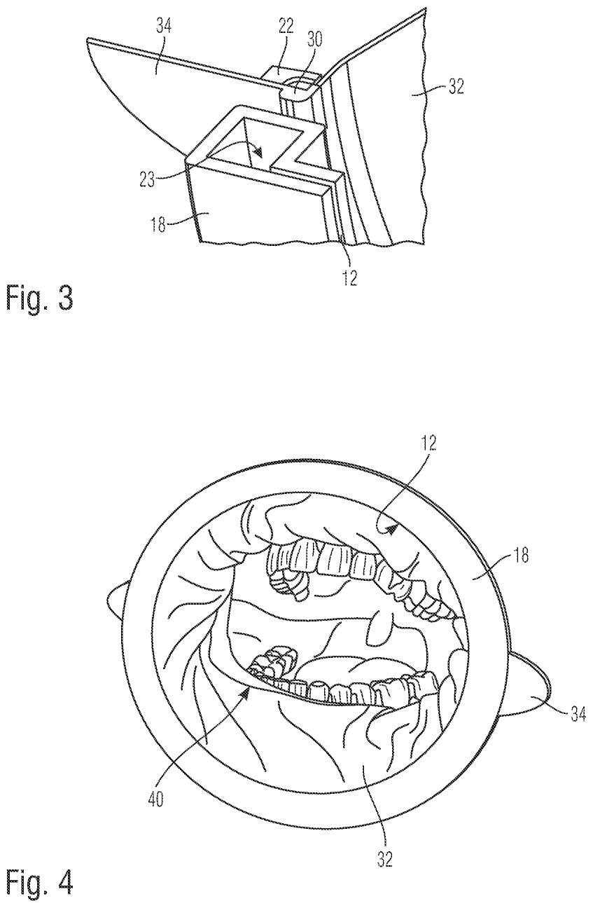

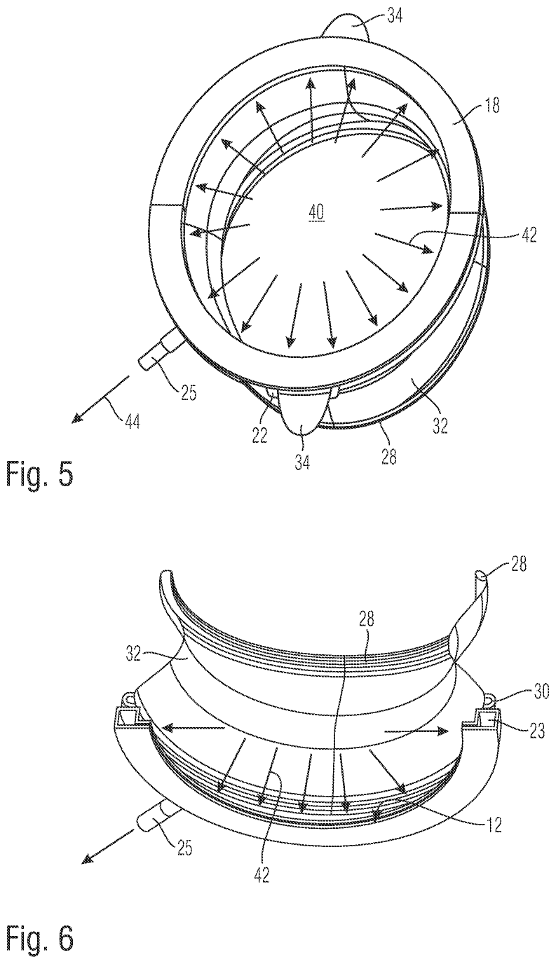

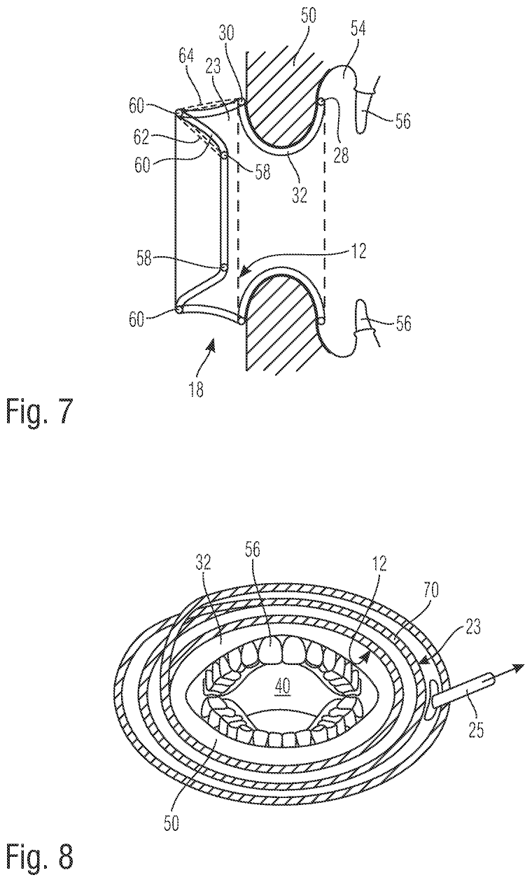

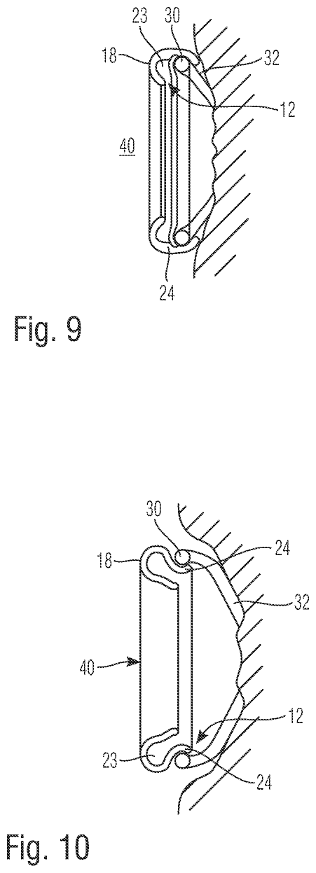

An enlarged illustration of a detail from FIG. 2 is shown in FIG. 3. Identical reference signs here as well as in the other FIGS. indicate identical or corresponding parts.

As can be seen in FIG. 3, the negative pressure chamber 23 is rectangular and in the example almost square. It is understood that any other, even a rounded negative pressure chamber 23 is possible instead. As can be seen in FIG. 3, the suction nozzle or area 12 extends as an annular gap. The length of the suction nozzle 12 in the direction of flow is significantly greater than the width of the annular gap. In the illustrated embodiment example, the suction nozzle 12 is aligned exactly radially inwards. It is understood that an orientation deviating from this can also be favourable instead.

In this way, the suction nozzle can be directed somewhat in the intraoral direction, i.e. in the direction of the film element 32. In this case, the main suction power is directed towards the patient's oral cavity, so that his breathing air is immediately extracted.

However, it is also possible to orient the suction nozzle 12 extraorally at an angle, i.e., pointing away from the film element 32. In this configuration, the breathing air is mainly extracted in front of the suction ring 10.

The deviation from the radial extension can be, for example, 5°, 10°, but also up to 45°.

It can also be seen from FIG. 3 that the tensioning flap 34 extends through the eyelet 22. In this embodiment example, the sleeve 22 also receives part of the tensioning ring 30 so that the latter is also clamped there.

FIG. 4 shows the use of a suction ring 10 according to the invention on a patient. The film element 32 covers the lips, with the tensioning element 28 extending in the vestibule.

The suction ring 10 extends extraorally and surrounds the patient's mouth, even when the mouth is open.

The suction tensioning element 18 holds the film element 32 taut, so that the tensioning element 30 is basically dispensable in terms of technical functions. However, it is convenient to use the tensioning element 30 for the bearing between the holder 26 and the suction tensioning element 18.

FIGS. 5 and 6 show the air flow 42 and 44 when using the suction ring 10 according to the invention. The air flow occurs initially radially outwards into the suction nozzle 12, then through the vacuum chamber 23 and via the suction port 25 to a vacuum source.

A further embodiment of a suction ring 10 according to the invention can be seen in FIG. 7. In this embodiment, the suction tensioning element 18 is formed by a circumferential ring similar to the tensioning rings 28/18 of the holder 26. In this solution, the vacuum chamber 23 is delimited by a foil corresponding to the film of the foundation 32.

The suction nozzle 12 is formed between the foremost/inner ring 58 of the tensioning element 18 and the film element 32 when the latter is in contact with the lip 50 of the patient. In FIG. 7, for clarity the patient's vestibule 54 and incisors 56 are also schematically indicated.

Here too, the suction ring 10 is circular, and FIG. 7 shows a vertical section through it, so that only part of the suction ring 10 is visible in the region of the upper jaw and part in the region of the lower jaw.

In the embodiment according to FIG. 7, the tensioning element 18 is designed as a structure consisting of several rings 30, 58 and 60. These rings are connected to each other via auxiliary bars 62 and 64, which are schematically indicated in FIG. 7. The connection is elastic, but in such a way that the negative pressure chamber 23 is kept taut.

In this embodiment, the rings 30, 58 and 60 basically form the suction tensioning element 18, together with the film extending between these rings.

A modified embodiment is shown in FIG. 8. In comparison with FIG. 7, the bars 62 and 64 are omitted. Instead, the suction tensioning element 18 is formed by a circumferential ring spiral 70.

The vacuum chamber 23 is also in communication with the suction port 25 in this solution, and the suction nozzle 12 extends between the innermost ring 58 and the film element 32.

This embodiment has the advantage that it is inexpensive to manufacture, using the same or a corresponding tool with which an “optragate” device can also be manufactured.

A further embodiment according to the invention can be seen in FIG. 9. This embodiment is only shown schematically. The film element 32 extends as shown in FIG. 7 and is attached to the tensioning ring 30.

The suction tensioning element 18 is provided with clips 24 as receiving means 20. The clips 24 overlap the tensioning element 30. They are part ring-shaped, but not as short as in FIG. 1, but extend, for example, over 45 degrees.

The tensioning ring 30 as part of the holder 26 is clipped into the clamps 24. This provides a secure connection to the tensioning element 18, but the tensioning element 18 of the suction ring 10 is readily detachable so that it can be reused.

The tensioning element 18 is a moulded plastic part. It has a rounded shape and forms the vacuum chamber 23 and the suction nozzle 12. The latter is aligned with the centre 40.

A similar embodiment can be seen in FIG. 10. Here, too, the tensioning element 18 is a moulded plastic part.

As in the embodiment according to FIG. 9, the tensioning element 18 is rounded and has neither inner corners nor sharp edges. This benefits the possibility of sterilisation or cleaning, but also reduces the risk of injury.

In contrast to the embodiment according to FIG. 9, the clamps 24 engage the tensioning element 30 from the inside. They spread it outwards, but also engage behind it, so that secure storage is guaranteed.

Here too, the holder 26 is interchangeable with the film element 32 and the tensioning elements 30 and 28, and the suction tensioning element 18 is reusable.

Although the invention is illustrated above, partly with reference to some preferred embodiments, it must be understood that numerous modifications and combinations of different features of the embodiments can be made. All of these modifications lie within the scope of the appended claims.

1. A suction ring for attachment close to the mouth opening of a dental patient comprising
an extraoral suction tensioning element, optionally in cooperation with a film, the suction tensioning element forms or spans a vacuum chamber, which vacuum chamber extends annularly or partially annularly, and
wherein the vacuum chamber has at least one suction nozzle, pointing radially inwardly.

2. The suction ring as claimed in claim 1,
wherein the suction tensioning element extends annularly around the suction nozzle and/or
wherein the suction nozzle extends annularly.

3. The suction ring as claimed in claim 2,
wherein the suction nozzle has a radially extending suction gap or a plurality of radially extending suction nozzles.

4. The suction ring as claimed in claim 1,
wherein the suction nozzle is configured for suction of gases comprising aerosols.

5. The suction ring as claimed in claim 1,
wherein the vacuum chamber is fixedly connected to a suction connection for connection to a vacuum source.

6. The suction ring as claimed in claim 1,
wherein the suction tensioning element has a round, circular, oval, or egg-shaped form in a top view from a forward side.

7. The suction ring as claimed in claim 1,
wherein the suction tensioning element has a receiver for the film element, via which it is connected or is configured to be connected, detachably, to the film element.

8. The suction ring as claimed in claim 1,
wherein the film element is firmly connected to the suction tensioning element and/or extends at least partially over the suction tensioning element.

9. The suction ring as claimed in claim 1,
wherein the suction tensioning element holds the film element in the tensioned state, and is placed under tension by the film element itself.

10. The suction ring as claimed in claim 1,
wherein the film element is configured to extend over a lip of a patient, at which point the film element forms a lip-covering element.

11. The suction ring as claimed in claim 1,
wherein the suction tensioning element is designed as an extraoral suction tensioning element which is connected to an intraoral tensioning element via a film element as a lip covering element.

12. The suction ring as claimed in claim 1,
wherein the suction tensioning element is connected or connectable to a lip/cheek holder or is formed integrally therewith.

13. The suction ring as claimed in claim 1,
wherein a lip/cheek holder is retrofitted with the suction ring.

14. The suction ring as claimed in claim 1,
wherein the vacuum chamber and/or the suction tensioning element is formed as a dimensionally stable plastic body, as an injection moulded part.

15. The suction ring as claimed in claim 1,
wherein the suction tensioning element is formed by or comprises a radially outwardly prestressed wire made of plastic or metal, and/or
wherein the suction tensioning element extends spirally, the spirals having different diameters, and/or
wherein the suction nozzle is formed between the cover element and the tensioning element, in the last spiral of the tensioning element.

16. The suction ring as claimed in claim 1 for use as a retrofit part or accessory part for a lip/cheek holder.
